(19) United States
(12) Patent Application Publication
(10) Pub. No.: US 2023/0044397 A1
(43) Pub. Date: Feb. 9, 2023

(54) FLUID LENS COMPONENT FOR INTRAOCULAR LENS AND METHODS OF PREPARING THE SAME

(71) Applicant: LensGen, Inc., Irvine, CA (US)

(72) Inventors: Thomas Silvestrini, Alamo, CA (US); Kim Van Thuy Nguyen, Santa Clara, CA (US); Richard S. Lilly, San Jose, CA (US); Richard James Ingram, Goleta, CA (US); Daniel Brady, San Juan Capistrano, CA (US)

(21) Appl. No.: 17/784,065

(22) PCT Filed: Dec. 9, 2020
(86) PCT No.: PCT/US2020/064083

§ 371 Date: Jun. 9, 2022

Related U.S. Application Data
(60) Provisional application No. 62/946,939, filed on Dec. 11, 2019.

(51) Int. Cl. A61F 2/16

(52) U.S. Cl. CPC A61F 2/1635; A61F 2250/0097; A61F 2250/0063; A61F 2220/005; A61F 2240/001; A61F 2002/169; A61F 2250/0003

(57) ABSTRACT

An intraocular device that includes a fluid lens is provided. The fluid lens has an anterior side and posterior side that can be coupled together to form a closed cavity therebetween that can be filled with a liquid material. A channel can be formed in the posterior side that is configured to receive a joining substance that bonds the anterior side to the posterior side and inhibit the unintended spread of the joining substance. A syringe can be extended through a port in the fluid lens to flow liquid material into the closed cavity. Subsequently, a plug can be inserted into the port to prevent the escape of the liquid material.

### INCORPORATION BY REFERENCE TO ANY PRIORITY APPLICATIONS

This application is a U.S. National Phase of International Application No. PCT/US2020/064083, filed Dec. 9, 2020, which claims the priority benefit of U.S. Provisional Application No. 62/946,939, filed on Dec. 11, 2019, which is hereby incorporated by reference in its entirety herein. Any and all applications for which a foreign or domestic priority claim is identified in the Application Data Sheet as filed with the present application are hereby incorporated by referenced under 37 CFR 1.57.

### BACKGROUND

### Field

This application relates to a fluid lens component configured to be coupled with an intraocular lens device and methods of preparing the same.

### Description of the Related Art

Surgical procedures on the eye have been on the rise as technological advances permit for sophisticated interventions to address a wide variety of ophthalmic conditions. Patient acceptance has increased over the last twenty years as such procedures have proven to be generally safe and to produce results that significantly improve patient quality of life.

Cataract surgery remains one of the most common surgical procedures, with over 28 million cataract procedures being performed worldwide per year. It is expected that this number will continue to increase as average life expectancies continue to rise. Cataracts are typically treated by removing the crystalline lens from the eye and implanting an intraocular lens (“IOL”) in its place. As conventional IOL devices are designed to provide clear distance visions, they fail to correct for presbyopia. As a result, reading glasses are still required. Thus, although the vision of patients who undergo a standard IOL implantation will not be clouded by the cataract, they are unable to change focus from far to near.

### SUMMARY OF THE INVENTION

Implanting an IOL in the eye of a patient that utilizes a fluid lens can enable a patient to change focus from far to near, improving the adaptability of the patient's vision. Care must be taken in preparing such a lens to maintain accommodative capacity, appropriate flexibility, and optical clarity as such may be compromised by contamination along the optical axis, and/or leakage of optical fluid.

Accordingly, there is a need for an IOL device that utilizes a fluid lens that can be prepared and placed in the capsular bag following capsulotomy and provide enhanced outcomes for patients. Enhanced outcomes can be in a variety of forms. For instance, accommodative IOL components (e.g., fluid lenses) disclosed herein have a channel that receives a joining substance to couple an anterior member and posterior member of the IOL components together. The channel and a proximate inclined interface between the anterior member and posterior member can restrict the joining substance to a predetermined area. Restricting the joining substance to a predetermined area reduces, minimizes or eliminates contamination of an optical fluid, which could degrade a patient's vision along the optical axis. Restricting the joining substance to the predetermined area also helps to maintain flexibility of accommodative components and the optical fluid such that the fluid lens avoids increased stiffness due to the unrestrained spread of the joining substance. Another enhanced outcome is the use of a plug to obstruct a port through which an optical fluid flows into a closed cavity of the IOL component. The plug can be configured to receive a syringe for filling a fluid space in the IOL component. The plug can reduce, minimize, or eliminate optical fluid leakage from the IOL component which can cause reduced accommodative capacity.

In some variants, a method of assembling a fluid lens is provided. An anterior member having an anterior optical surface and a first peripheral portion is provided. A posterior member having a posterior optical surface and a second peripheral portion is provided. A joining substance is applied to one or both of an anterior surface of the second peripheral portion or a posterior surface of the first peripheral portion. The anterior surface of the second peripheral portion is coupled with the posterior surface of the first peripheral portion using the joining substance. A closed cavity is formed between the anterior optical surface and the posterior optical surface by forming a seal between the first peripheral portion and the second of the peripheral portion.

In some variants, applying a joining substance can include flowing a liquid material into a channel formed in the anterior surface of the second peripheral portion or into a channel formed in the posterior surface of the first peripheral portion.

In some variants, applying a joining substance includes flowing a liquid material into a channel formed in the anterior surface of the second peripheral portion.

In some variants, the method can include enclosing the channel to block flow of the joining substance into the closed cavity. In some variants, enclosing the channel can include engaging an inclined interface formed between a second inclined surface of the second peripheral portion and a first inclined surface of the first peripheral portion.

In some variants, the method can include confirming that the closed cavity is substantially free of the joining substance. In some variants, the method can include detecting a visible color mixed with the joining substance.

In some variants, an intraocular lens component is provided. The intraocular lens component has an anterior side having an anterior optical surface disposed across an optical axis of the lens component. The intraocular lens component has a posterior side having a posterior optical surface disposed across the optical axis. The intraocular lens component has a peripheral portion having an anterior portion coupled to the anterior side. The intraocular lens component has a posterior portion coupled to the posterior side. The intraocular lens component has a joining channel disposed in one or both of the anterior portion and the posterior portion. The intraocular lens component has a joining substance disposed in the joining channel to bond the posterior portion of the peripheral portion to the anterior portion of the peripheral portion.

In some variants, the joining substance includes a pigment or dye that can facilitate visualization of an orientation of an intraocular lens. In some variants, the pigment or dye is closer to the anterior side or posterior side.

In some variants, the joining substance forms a continuous feature in the peripheral portion that is configured to visually verify assembly of an intraocular lens when viewed from the anterior side. In some variants, the continuous feature is an annular structure that surrounds the optical axis of the lens component. In some variants, the continuous feature can be visually disrupted under a retaining feature of the intraocular lens to visually verify the assembly of the intraocular lens when viewed from the anterior side.

In some variants, the intraocular lens component includes an inclined interface disposed in the peripheral portion between the posterior portion and the anterior portion. In some variants, the inclined interface has an anterior inclined surface and a posterior inclined surface, the inclined interface being disposed between the joining channel and a closed cavity. In some variants, the closed cavity is between the anterior optical surface and the posterior optical surface.

In some variants, the intraocular lens component includes a material that can adhere to a surface of a base member upon contact. In some variants, an anterior surface of the anterior portion, a posterior surface of the posterior portion, and a peripheral surface of the peripheral portion are rough to facilitate improved adhesion to the surface of the base member upon contact. In some variants, the peripheral surface of the peripheral portion is rough to facilitate improved adhesion to the surface of the base member upon contact.

In some variants, an intraocular lens device includes an intraocular lens component and a base member. The base member can have a haptic that can engage a capsular bag of an eye of a patient. The base member can have one or more retention features. The base member can include a material that can adhere to a surface of the IOL component upon contact. In some variants, the base member can have a radially inward facing wall, posterior surface of the one or more retention members, and/or one or more tables. The base member can have a receiving space that is adjacent the radially inward facing wall and/or between the one or more retention members and the one or more tables. The receiving space can receive the IOL component. The radially inward facing wall, posterior surface of the one or more retention members, and/or one or more tables can be rough to facilitate improved adhesion to surface(s) of the IOL component upon contact.

In some variants, a method of preparing a fluid lens component is provided. A fluid lens component having a closed cavity disposed between an anterior optical member, a posterior optical member, and a peripheral portion is provided. The peripheral portion has a peripheral surface having a port extending toward the closed cavity. The anterior optical member has a first surface on an anterior side of the fluid lens and a second surface opposite the first surface. The second surface is at least partially bounding the closed cavity. A tubular member is advanced into and through the port such that the tubular member extends into the closed cavity. An optical fluid is dispensed in the closed cavity to provide a continuous expanse of the optical fluid adjacent to the second surface of the anterior optical member. The tubular member is withdrawn from the port.

In some variants, the fluid lens component has a solid wall disposed between an inward end of the port and the closed cavity. In some variants, advancing the tubular member includes piercing the solid wall to provide access to the closed cavity through the port.

In some variants, dispensing the optical fluid includes positioning a tip of the tubular member at a side of the closed cavity opposite the port and withdrawing the tip as the fluid is flowing into the closed cavity. In some variants, the method includes forming a plug in the port after withdrawing the tubular member from the port.

In some variants, an intraocular lens component is provided. The intraocular lens component includes an anterior side including an anterior optical surface disposed across an optical axis of the lens component. The intraocular lens component has a posterior side having a posterior optical surface disposed across the optical axis. The intraocular lens component includes a peripheral portion coupled to the anterior side. The posterior side, the peripheral portion, and anterior side form a closed cavity therebetween. The port has a first end disposed at a peripheral surface of the peripheral portion and a second end disposed within the peripheral portion between the first end and the closed cavity.

In some variants, a solid and continuous expanse of material extends between the second end of the port and the closed cavity. In some variants, the intraocular lens component includes a channel formed between the second end of the port and the closed cavity for access to the closed cavity by a syringe. The channel is self-sealing in the absence of the syringe. In some variants, the intraocular lens component includes an optical fluid disposed in the closed cavity. In some variants, the intraocular lens component includes a plug member disposed in the port.

In some variants, an intraocular lens component is provided. The intraocular lens component has an anterior side having an anterior optical surface disposed across an optical axis of the intraocular lens component. The intraocular lens component has a posterior side having a posterior optical surface disposed across the optical axis. The intraocular lens component includes a peripheral portion coupled to the anterior side and the posterior side. A fluid-filled cavity extends between the anterior optical surface, posterior optical surface, and peripheral portion. The intraocular lens component has a base member including a haptic that can engage a capsular bag of an eye of a patient and one or more resilient retention features having an inner end biased posteriorly into a receiving space configured to receive the (IOL) component. The one or more resilient retention features can be moved in an anterior direction to facilitate placing the IOL component into the receiving space of the base member and to be released to be moved toward an anterior surface of the peripheral portion of the IOL component.

In some variants, the resilient retention feature can, upon being released after the IOL component is disposed in the base member, apply a normal force on the anterior surface of the peripheral portion of the IOL component.

In some variants, the IOL component has a notch that can receive one or more of the resilient retention features when the IOL component is at a pre-defined rotational position relative to the base member.

In some variants, the IOL component has an anterior-posterior thickness that is larger than an anterior-posterior thickness of the receiving space such that the IOL component causes the one or more resilient retention features to flex anteriorly upon assembly of the IOL component within the base member.

In some variants, a method of assembling an intraocular lens (IOL) device within an eye of a patient is provided. A base member having a base optic and one or more resilient retention features that have an inner end biased posteriorly toward the base optic is inserted into an eye of a patient. The one or more resilient retention features are moved anteriorly relative to a free state of the resilient retention feature. The intraocular lens (IOL) component is inserted into a receiving space between the one or more resilient retention features and an opposite portion of the base member. The one or more resilient retention features are released such that the one or more resilient retention features contact an anterior surface of the IOL component.

In some variants, releasing the one or more resilient retention features can result in the one or more resilient retention feature(s) moving into a notch in the IOL component.

In some variants, the one or more resilient retention features applies a normal force to the anterior surface of the IOL component.

In some variants, the method further includes contacting a peripheral surface of the IOL component with a radially inward facing surface of the base member.

In some variants, the anterior surface of the IOL component anteriorly deflects the one or more resilient retention features.

### BRIEF DESCRIPTION OF THE DRAWINGS

These and other features, aspects and advantages are described below with reference to the drawings, which are intended for illustrative purposes and should in no way be interpreted as limiting the scope of the embodiments. Furthermore, various features of different disclosed embodiments can be combined to form additional embodiments, which are part of this disclosure. In the drawings, like reference characters denote corresponding features consistently throughout similar embodiments. The following is a brief description of each of the drawings.

FIG. 1 shows an anterior perspective, broken out view of an eye with an intraocular lens (IOL) device according to one embodiment of the present disclosure disposed therein;

FIG. 2 is an anterior perspective view of the IOL device shown in FIG. 1;

FIG. 3 is an exploded view of the IOL device of FIG. 2;

FIG. 4 is an anterior perspective view of an IOL component;

FIG. 5 is a top view of the IOL component shown in FIG. 4;

FIG. 6 is a sectional view of an IOL component of the IOL device shown in FIG. 1.

FIG. 7 is an enlarged view of a portion of the IOL component shown in FIG. 6.

FIG. 7-1 is an enlarged view of a portion of the IOL component shown in

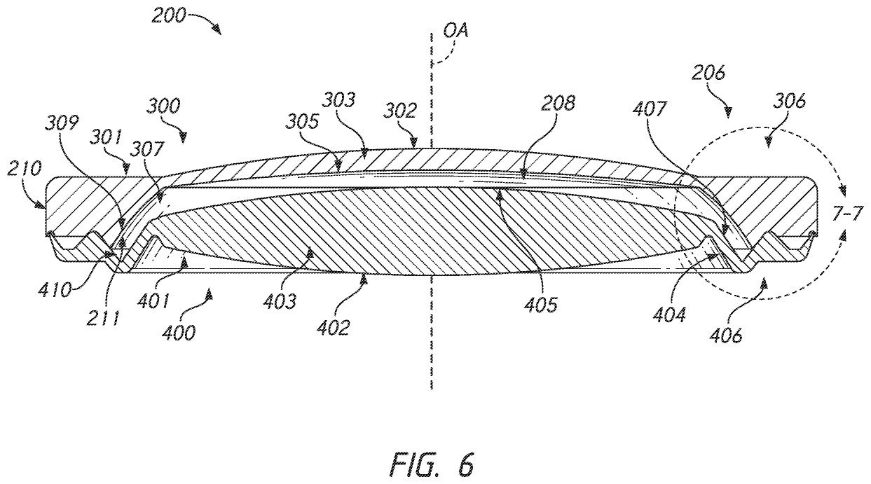

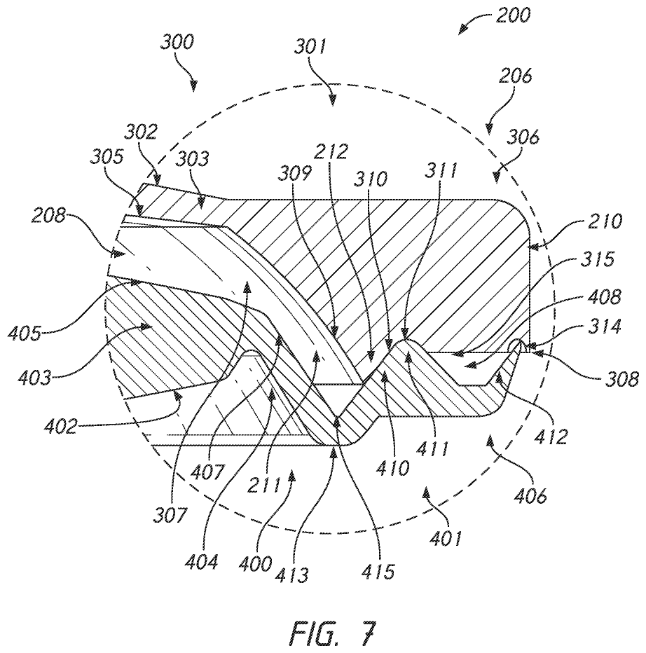

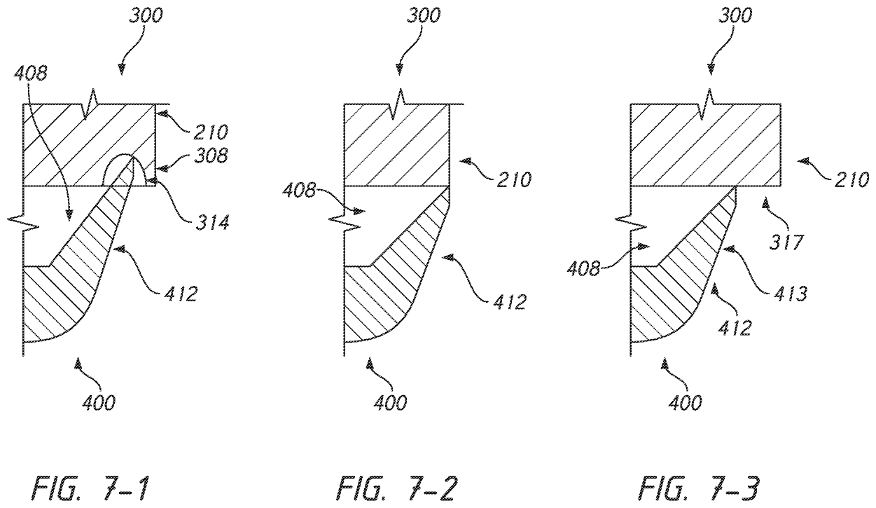

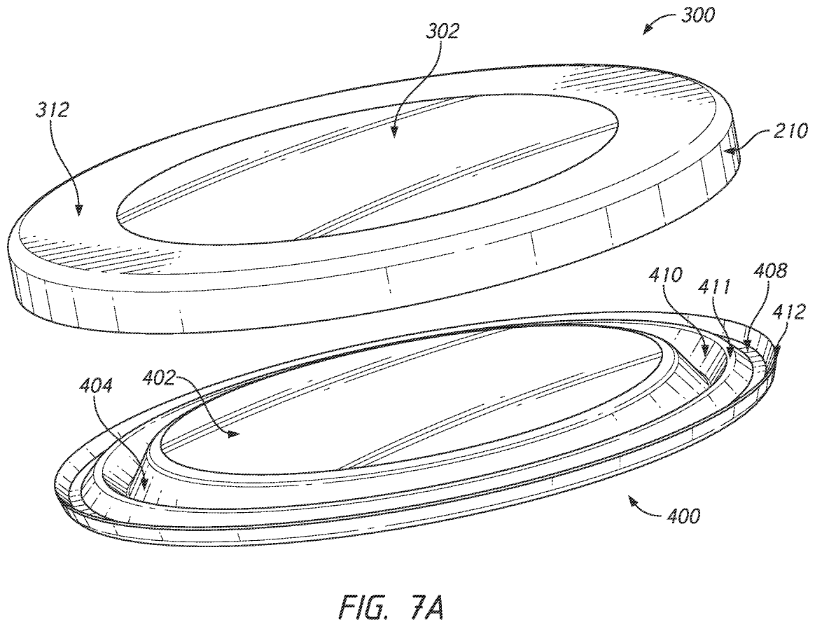

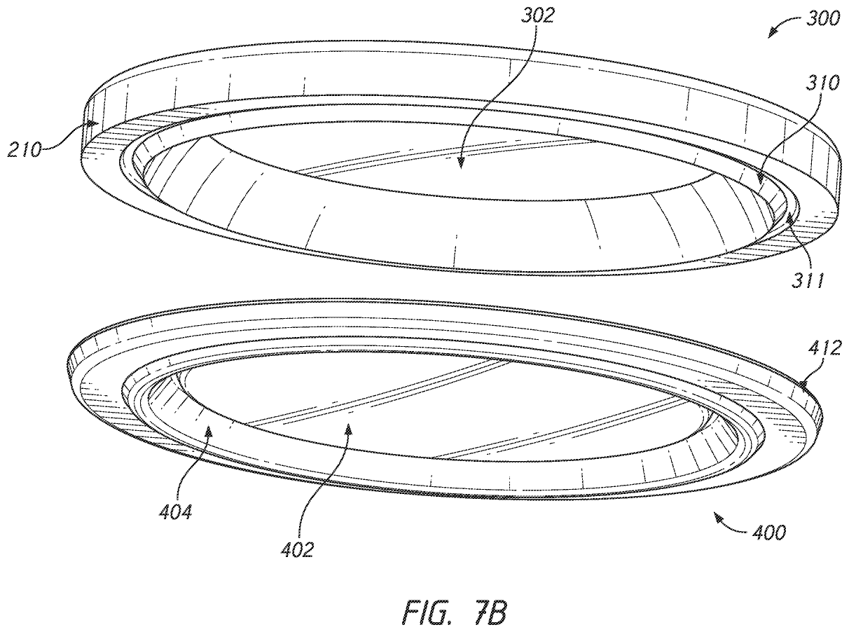

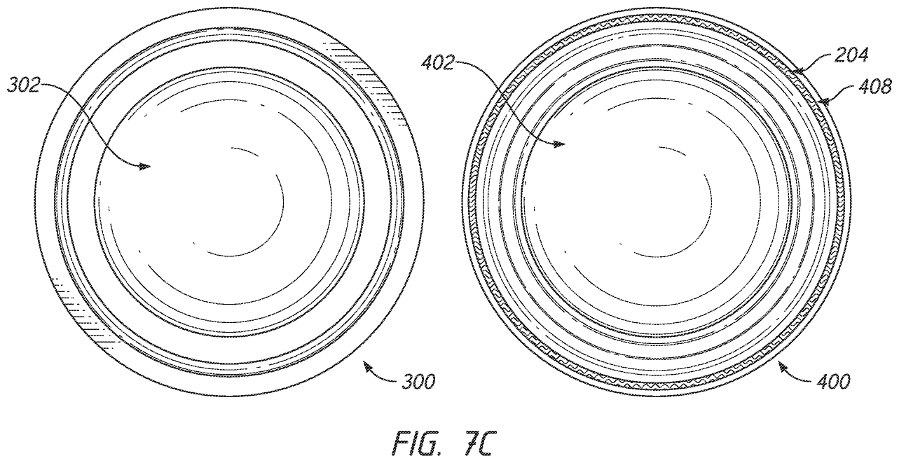

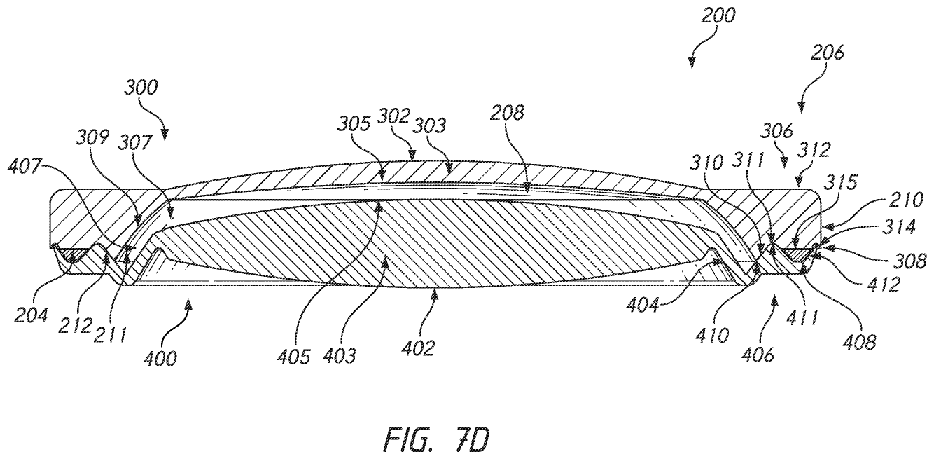

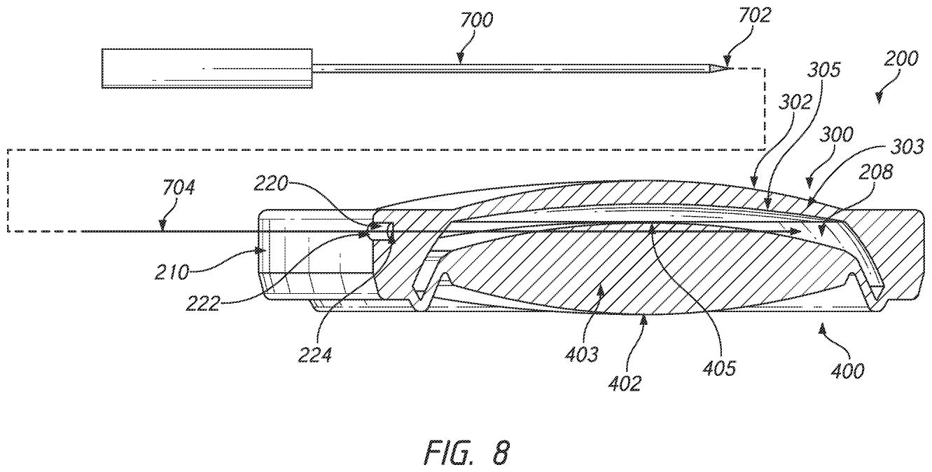

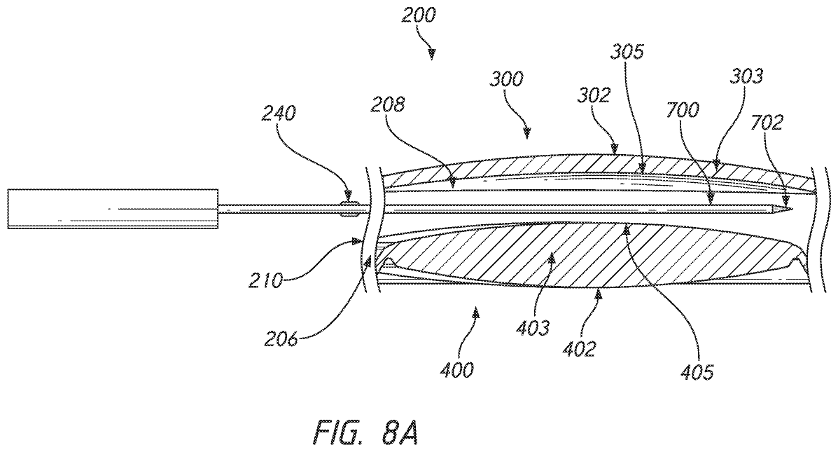

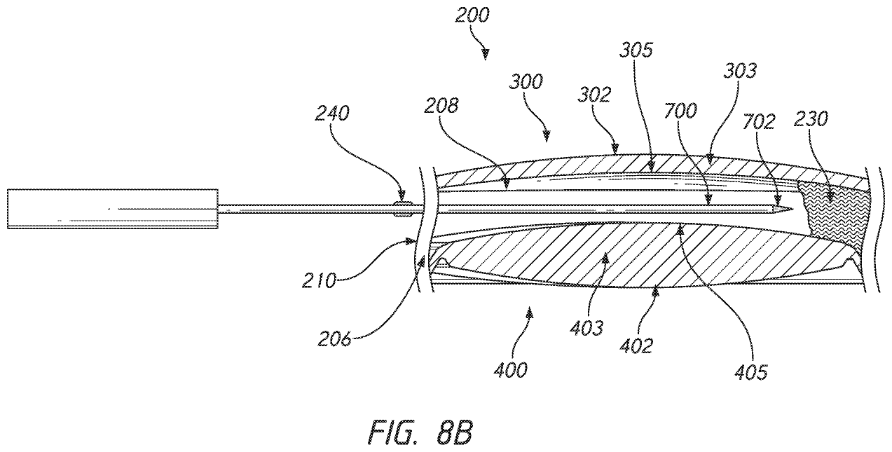

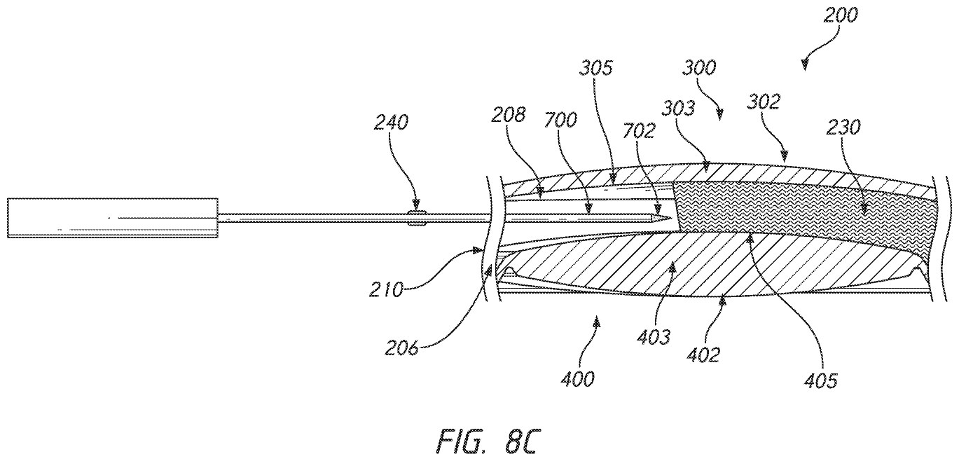

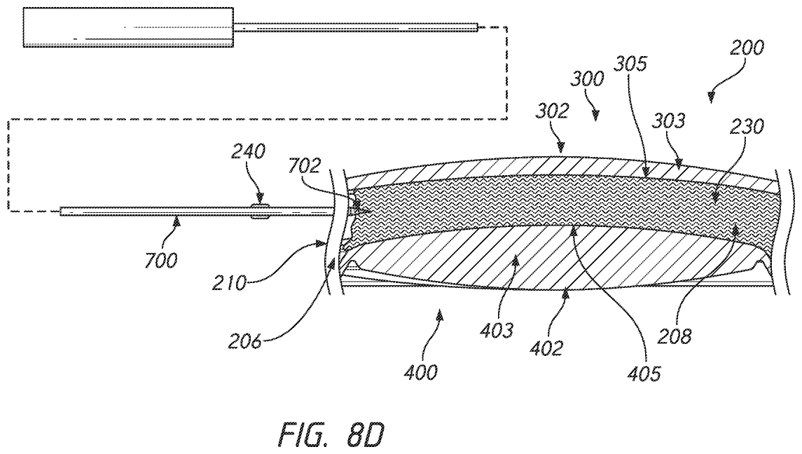

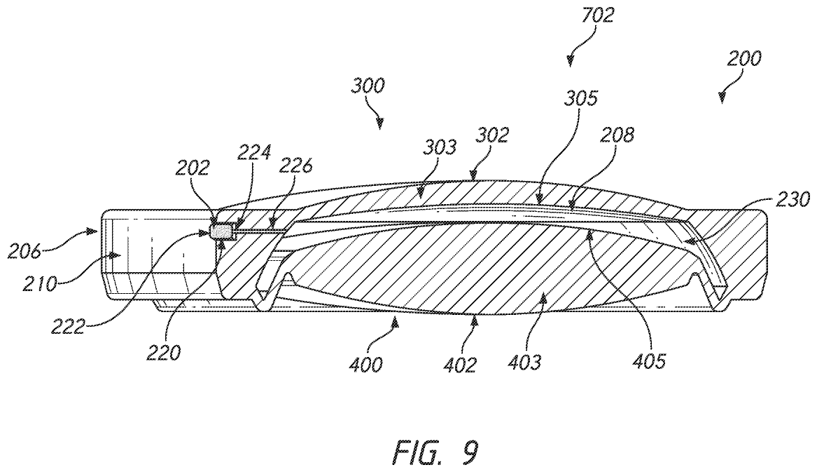

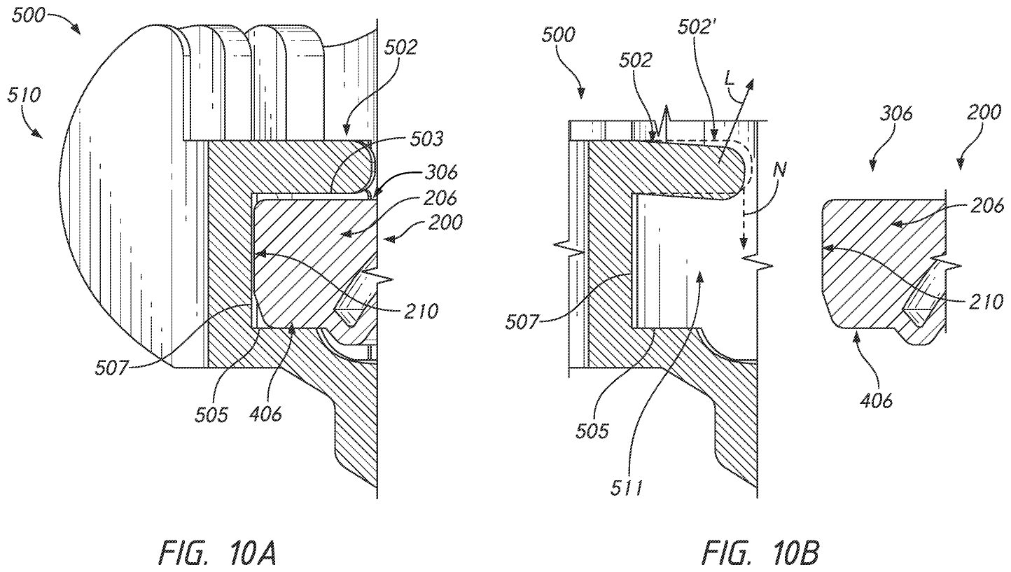

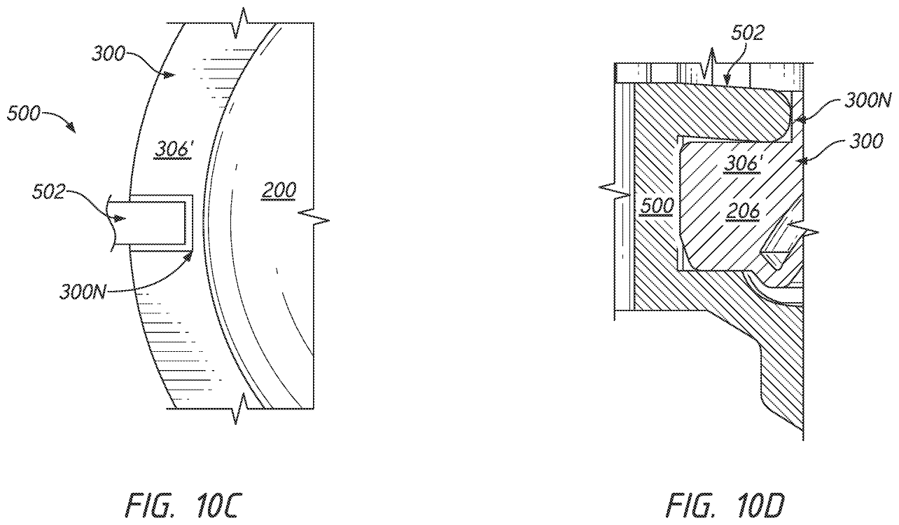

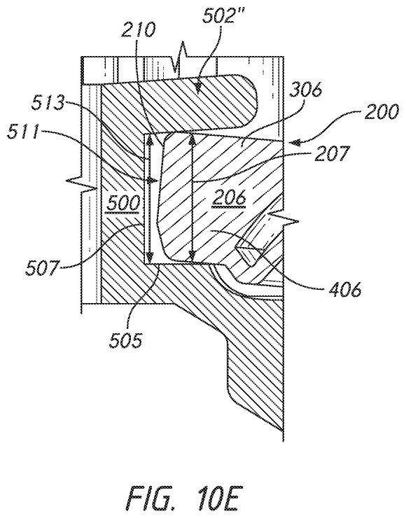

FIGS. 6 and 7 with a recess for coupling with an arm.

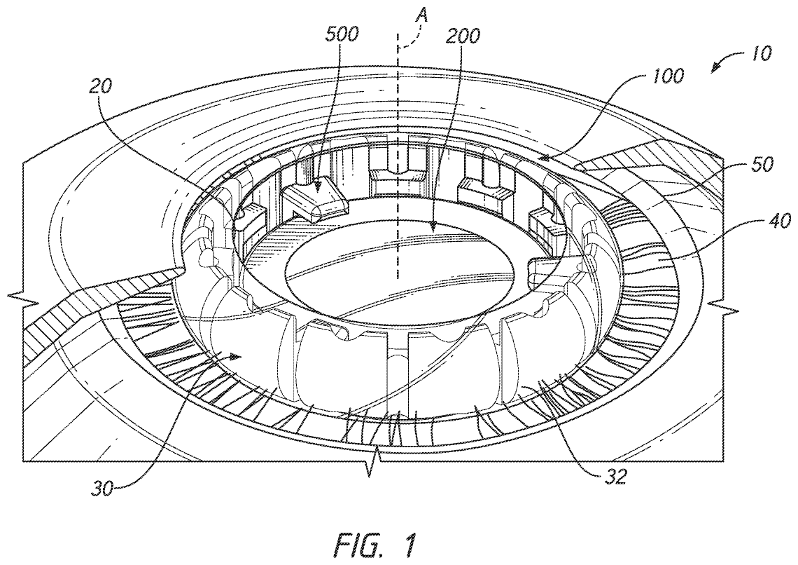

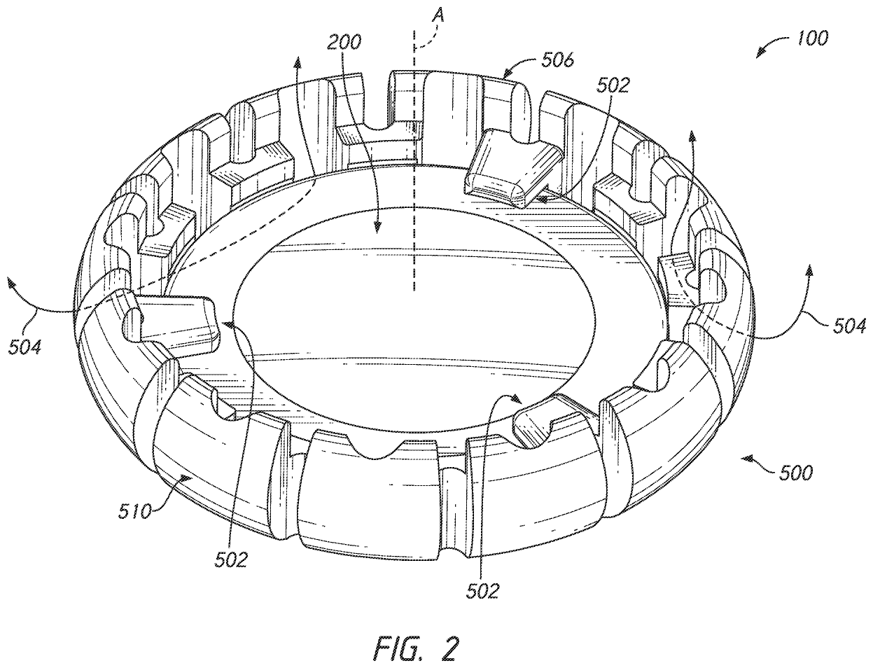

FIG. 7-2 is an enlarged view similar to that of FIG. 7-1 showing a portion of an IOL component having an arm that engages with a substantially flat posterior surface of an anterior member of the IOL component.

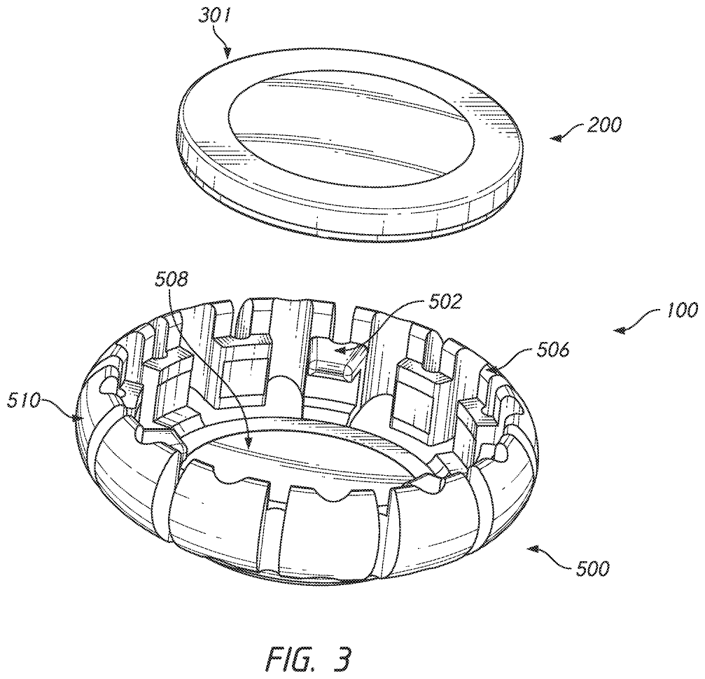

FIG. 7-3 is an enlarged view similar to that of FIG. 7-2 showing an arm of a posterior member engaging a posterior surface of the anterior member at a position that is removed from, e.g., radially inward of, a peripheral surface of the IOL component.

FIG. 7A is an exploded anterior perspective view of the IOL component shown in FIG. 6.

FIG. 7B is an exploded posterior perspective view of the IOL component shown in FIG. 6.

FIG. 7C is a view of the disassembled IOL component shown in FIG. 6 with a channel filled with a joining substance in an anterior surface of the posterior side.

FIG. 7D is a sectional view of the IOL component shown in FIG. 6 with the joining substance in the channel.

FIG. 8 is a sectional view of an IOL component with a tubular member.

FIG. 8A is a sectional view of the IOL component of FIG. 8 with the IOL component compressed and the tubular member advanced into and through a port to a far side of the closed cavity.

FIG. 8B is a sectional view of the IOL component of FIG. 8 with the IOL component compressed and the tubular member being withdrawn while flowing liquid material into the closed cavity.

FIG. 8C is a sectional view of the IOL component of FIG. 8 with the IOL component compressed and the tubular member being withdrawn while flowing liquid material into the closed cavity.

FIG. 8D is a sectional view of the IOL component of FIG. 8 with the IOL component compressed and the tubular member being withdrawn from the port with the closed cavity filled with liquid material.

FIG. 9 is a sectional view of the IOL component of FIG. 8 with a plug inserted into the port of the IOL component.

FIG. 10A is a portion of a sectional view of an IOL as illustrated in FIGS. 1-2 showing the IOL component of FIG. 8 inserted into a base member.

FIG. 10B is an exploded and sectional view of the base member of FIG. 10A with a resilient retention feature at different positions under varying loads.

FIGS. 10C and 10D are anterior side and cross-sectional views of a portion of an IOL including an IOL component with a notch to facilitate rotational positioning.

FIG. 10E is a portion of a sectional view of an IOL component anteriorly deflecting a resilient retention feature of a base member.

### DETAILED DESCRIPTION

This application discloses a multi-component IOL device 100 (e.g., accommodating IOL device, accommodating IOL). The multi-component IOL device 100 has an IOL component 200 (e.g., a fluid lens, a power changing lens). The IOL component 200 has an anterior member 300 and a posterior member 400 that are joined together via a joining substance 204. A closed cavity 208 is provided between the anterior and posterior members 300, 400 that can receive a liquid material 230. Various configurations of the IOL component 200 and methods of preparing the same are disclosed herein.

### I. Intraocular Lens Device Embodiments

Providing clear focus is one of the chief aims of surgery in the front part of the eye. The IOL device 100 described herein achieves this objective and in addition is adapted to provide clear focus not only at distance but also up close, e.g., from near to far, in various embodiments. Thus the IOL device 100 is uniquely configured to provide premium performance for patients.

A. Eye Anatomy and Accommodation

FIG. 1 shows an eye 10 following placement of the IOL device 100. The natural lens of the eye 10 has been modified by a capsulotomy procedure in which an opening 20 is formed in the natural crystalline lens and the capsular bag 30 is evacuated of its contents through the opening 20. The opening 20 provides access through an access pathway from an exterior of the eye 10 for placement of the IOL device 100 in the capsular bag 30.

An equatorial region 32 of the capsular bag 30 is coupled to a ciliary muscle 50 by zonules 40. The zonules 40 are connective tissues that can stretch the capsular bag 30. When the ciliary muscle 50 is in a rest state, the zonules 40 are stretched and apply a tension force to the capsular bag 30. When the eye 10 attempts to accommodate, the ciliary muscle 50 contracts, reducing the tension in the zonules 40. These accommodation processes result in a compression force on a base member 500 of the IOL device 100 as discussed further below. Without being bound to a particular theory, it is believed that the capsular bag 30 contracts when the tension in the zonules 40 is reduced and the contraction applies a compression force to the base member 500 to cause accommodation of an IOL component 200 within the base member 500. The ocular forces of the eye 10 are sufficient to change the shape of one or more optical surface of the IOL component 200, resulting in accommodation.

B. Separate Component Intraocular Lens Structure

FIGS. 2-3 show the IOL device 100 shown in FIG. 1 assembled and disassembled separate from the eye 10. The IOL device 100 includes the base member 500 and the IOL component 200. The IOL component 200 is separate from the base member 500, as shown in FIG. 3, such that the base member 500 and the IOL component 200 can be delivered separately, e.g., sequentially into the eye 10 of a patient. The base member 500 can be delivered before the IOL component 200. The IOL component 200 can be subsequently delivered into the base member 500 and unfolded within the base member 500 while the base member 500 is in the capsular bag 30 of an eye 10. In some embodiments, the IOL component 200 and/or base member 500 can be made of a material with adhesive qualities (e.g., tacky, sticky) that adheres the surface(s) of the IOL component 200 to a structure disposed outward of the component 200, e.g., to the surface(s) of the base member 500 upon contact, facilitating improved retention of the IOL component 200 to the base member 500 or other structure configured or positioned to hold the component 200. In some embodiments, the surfaces of the IOL component 200 that interface with the base member 500 and/or the surfaces of the base member 500 that interface with the IOL component 200 can have adhesive qualities (e.g., tacky, sticky) that adhere the surface(s) of the IOL component 200 to the surface(s) of the base member 500 upon contact. In some embodiments, the material is tacky.

The base member 500, as shown in FIG. 3, includes a base lens 508 and a haptic 510. The base lens 508, if present, provides some of the focusing power of the IOL device 100, which can include fixed or static focusing power. The haptic 510 can extend into the equatorial region 32 of the capsular bag 30 and establish a mounting position for the IOL component 200. The base member 500 has an opening 506 that can be at an opposite end of the base member 500 relative to the base lens 508. The IOL component 200 can be inserted through the opening 506 and positioned behind retaining features 502 (e.g., lens retention members, tabs, protrusions, retainers, etc.), as shown in FIG. 2, enabling the IOL component 200 to be assembled with and/or coupled to the base member 500. The retaining features 502 can be positioned anterior to the base lens 508 of the base member 500 when present. When viewed anteriorly, the retaining features 502 can be positioned anterior to the IOL component 200 when the IOL component 200 is assembled with and/or coupled to the base member 500. Even when assembled, as shown in FIG. 2, the IOL device 100 can allow fluid to flow through fluid pathways 504 between the anterior and posterior sides of the IOL device 100 and/or between the base lens 508 and posterior side of the IOL component 200.

### II. Intraocular Lens Components

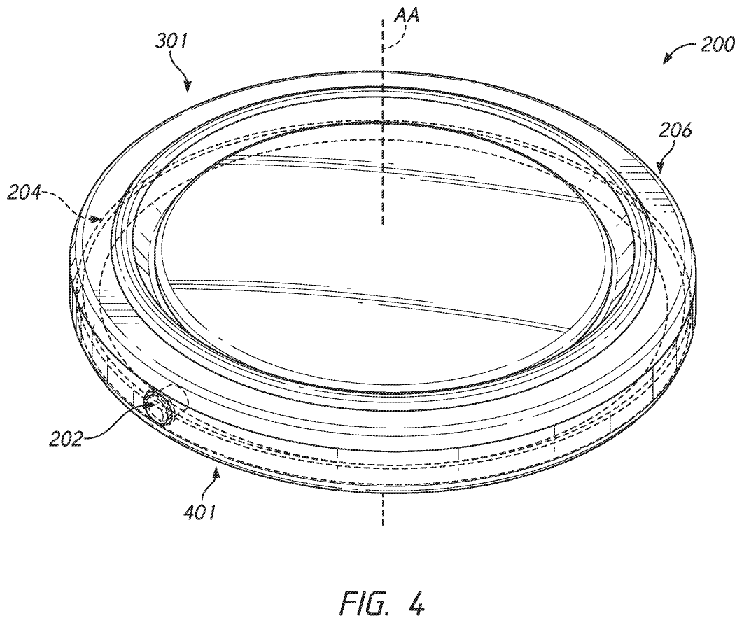

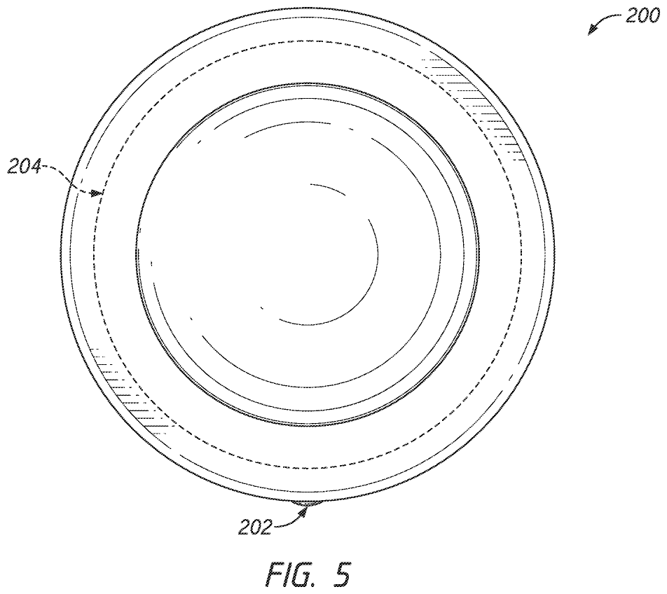

FIGS. 4-5 show one embodiment of an IOL component 200. The IOL component 200 has at least two sides, an anterior side 301 (also called a first side) and a posterior side 401 (also called a second side) (also shown in FIGS. 6-7D) that are joined together by a joining substance 204 (e.g. a bonding agent such as an adhesive). The anterior side 301 and the posterior side 401 are disposed across the optical axis AA of the IOL component 200. The joining substance 204 can be made of the same material as the IOL component 200 and/or another suitable material.

The joining substance 204 can include a pigment and/or dye such that the joining substance 204 has a color (e.g., yellow) that is visible. The intensity of the color can impact visibility with higher intensity colors being more visible than lower intensity colors. In some embodiments, the joining substance 204 can be opaque, which can include being non-transmissive of less than 10%, 10%, 20%, 30%, 40%, 50%, 60%, 70%, 80%, 90%, or greater than 90% of light thereon. In some embodiments, the joining substance 204 can be reflective, which can include reflecting less than 10%, 10%, 20%, 30%, 40%, 50%, 60%, 70%, 80%, 90%, or greater than 90% of light thereon. In some embodiments, the joining substance 204 can be fluorescent such that the joining substance 204 emits light at a particular wavelength after absorbing light at another wavelength. In some embodiments, the joining substance 204 emits light when exposed to specific wavelengths and, upon removal of the light source, ceases or almost immediately ceases to emit light. This can advantageously allow for selective visibility or intensity of visibility based upon exposure to specific light sources. Accordingly, the joining substance 204 can be exposed to a specific light source such that light is selectively emitted during assembly of the IOL component 200 or IOL device 100 but not emitted, absent the specific light source, during normal use after implantation in the eye of a patient. For example, fluorescence of the joining substance 204 can occur when the absorbed radiation is ultraviolet light (invisible to the human eye) while the emitted light is a distinct color in the visible region. After removal of the ultraviolet light source, the fluorescence of the joining substance 204 can almost immediately cease. In some embodiments, the joining substance 204 is phosphorescent and continues to emit light for a duration of time after a radiation source is removed.

The pigment and/or dye can facilitate visualization of an orientation of the IOL device 100 and/or IOL component 200 and/or relative position of one or more components of the base member 500 or the IOL component 200. The pigment and/or dye of the joining substance 204 can be closer to the anterior side 301 or posterior side 401. Stated differently, the joining substance 204 can be closer to the anterior side 301 or posterior side 401. For example, as shown in FIG. 4, the pigment and/or dye of the joining substance 204 is closer to the posterior side 401 of the IOL component 200 than to the anterior side 301 thereof, enabling the orientation of the IOL component 200 to be determined when viewed from the side. One can confirm that the joining substance 204 of the IOL component 200, made visible by the color (e.g., yellow color) thereof, is located posterior of a mid-plane of the IOL component 200 for example. In one variation the joining substance is closer to the anterior side 301 than to the posterior side 401 and as such one can confirm that the portion colored (e.g., yellow) by the joining substance 204 of the IOL component 200 is located anterior of a mid-plane of the IOL component 200 when confirming the orientation of the IOL component 200 is as intended.

In some aspects, the joining substance 204 can form a continuous feature in the peripheral portion 206 of the IOL component 200 that can be used to visually verify assembly of the IOL device 100 when viewed from the anterior side 301. The continuous feature can be an annular structure, which can surround an optical axis AA of the IOL component 200. The continuous structure can aid in confirming proper integration of the IOL component 200 with the base member 500. For example, the continuous feature can be visually disrupted under a retaining feature 502 of the base member 500 to visually verify the proper assembly of the IOL component 200 within the base member 500 when viewed from the anterior side of the IOL device 100. In other words, a visual inspection of a properly assembled IOL device 100 would provide at least two, e.g., three, elongate colored (e.g., yellow) arcs interrupted by at least two, e.g., three, relatively short circumferential spans of the retaining features 502 of the base member 500.

The IOL component 200 can have a closed cavity 208 (shown in FIGS. 6 and 7) that contains a liquid material (e.g. fluid, optical fluid, lens oil) and/or a gel material. Fluid can be inserted into the closed cavity 208 in any suitable manner, e.g., through a port in a periphery of the IOL component 200. In some cases, such a port can be sealed with a sealing component, such as a plug 202, that can be added following filling to prevent the fluid or gel from flowing out of the closed cavity 200.

FIG. 6 shows a cross-section of the IOL component 200. An anterior member 300 and a posterior member 400 of the IOL component 200 are interfacing with each other. The anterior side 301 is the anterior surface of the anterior member 300. The posterior side 401 is the posterior surface of the posterior member 400. The anterior member 300 has an anterior optical member 303 (e.g., membrane, flexible membrane, deformable membrane, first optical member). The anterior optical member 303 has an anterior optical surface 302 (e.g., first optical surface, first surface) on the anterior side of the fluid lens component 200, anterior member 300, and anterior optical member 303. The anterior optical member 303 has a second surface 305 (e.g. posterior optical surface of the anterior optical member 303) opposite the anterior optical surface 302. The second surface 305 is the posterior surface of the anterior optical member 303.

The posterior member 400 has a posterior optical member 403 (e.g., posterior optic, second optical member, lens body). In one embodiment, the posterior optical member 403 is a biconvex lens, which can have a fixed power. The posterior optical member 403 has a posterior optical surface 402 (e.g., second optical surface) on the posterior side of the fluid lens component 200, posterior member 400, and posterior optical member 403. The posterior optical member 403 has a first surface 405 (e.g. anterior optical surface of the posterior optical member 403) opposite the posterior optical surface 402. The first surface 405 is the anterior surface of the posterior optical member 403.

The anterior optical member 303 is spaced away from the posterior optical member 403. The anterior optical surface 302 is spaced away from the posterior optical surface 402. The anterior optical surface 302 is disposed away from the posterior optical surface 402 in a free state. When a compressive force is applied to the periphery portion 206 of the IOL component 200, the configuration of the IOL component 200 can be changed to provide a higher optical power. For example, the anterior optical surface 302 can bend such that the surface of the anterior optical surface 302 is steeper, providing greater power. In some embodiments, movement of the anterior optical surface 302 relative to the posterior optical surface 402 can result in a change of (e.g., greater) power. In some examples a combination of relative movement between the surfaces 302, 402 and bending of the surface 302 provides a change in (e.g., greater) power. In some variants, the posterior optical surface 402 moves anteriorly when a compressive force is applied to the peripheral portion 206 of the IOL component 200. In some variants, the second optical surface 402 does not substantially move as a result of compression.

The compression of the IOL component 200 can arise when the peripheral surface 210 of the peripheral portion 206 is engaged with the base member 500 and when compressive forces applied to the haptic 510 results in compressive forces being applied to the peripheral surface 210, causing the IOL component 200 to accommodate.

The anterior member 300 including the anterior optical surface 302 is spaced apart from the posterior member 400 including the posterior optical surface 402 by a closed cavity 208 that can be filled with a liquid or gel material. Stated differently, the closed cavity 208 is between the anterior optical surface 302 and the posterior optical surface 402. The closed cavity 208 is between the anterior optical member 303 and the posterior optical member 403. The closed cavity 208 also is between an anterior optical surface of the posterior member 400 and a posterior optical surface of the anterior member 300. In some variants, the anterior optical surface 302 and the posterior optical surface 402 are spaced apart from each other in both an unaccommodated and accommodated state. The liquid or gel material in the closed cavity 208 can move towards or away from central and peripheral regions of the closed cavity 208 as the shape and/or curvature of the closed cavity 208 and anterior optical surface 302 change during accommodation. In some embodiments, the liquid or gel material is displaced as a result of an action on the anterior member 300 (e.g., bending of the optical surface 302) or the posterior member 400 (shifting of the position of the posterior surface 402 along the optical axis OA).

The anterior member 300 and the posterior member 400 are joined together by the peripheral portion 206, as shown in FIGS. 6 and 7. The anterior member 300 has a first peripheral portion 306 (e.g., anterior peripheral portion) forming part of the peripheral portion 206. The posterior member 400 has a second peripheral portion 406 (e.g., posterior peripheral portion) forming part of the peripheral portion 206. Stated differently, the peripheral portion 206 of the IOL component 200 has a first peripheral portion 306 and a second peripheral portion 406.

As shown in FIG. 7, the anterior optical surface 302 can be coupled to the peripheral portion 206 and first peripheral portion 306. The anterior optical surface 302 can be directly coupled to the peripheral portion 206, which can enable forces applied to the peripheral portion 206 to be directed to the anterior optical surface 302 to cause a change in shape and/or curvature. The posterior optical surface 402 can be coupled to the peripheral portion 206 and second peripheral portion 406 via a posterior coupler 404. The posterior coupler 404 can be angled toward the anterior optical surface 302 such that a lens body 403 of the posterior member 400 is disposed within a cavity 307 (e.g., a concave recess) of the anterior member 300. The lens body 403 can be partially and/or fully disposed in the cavity 307. The posterior coupler 404 can connect to the posterior optical member 403 at a position that is closer to the anterior surface 405 of the posterior optical member 403 than the posterior optical surface 402. In some embodiments, the posterior coupler 404 can connect to the posterior optical member 403 closer to the posterior optical surface 402 or at a midpoint between the anterior surface 405 of the posterior optical member 403 and the posterior optical surface 402.

The cavity 307 of the anterior member 300 is bounded by the posterior surface 305 of the anterior optical member 303 and an inner peripheral surface 309. The inner peripheral surface 309 can be curved and posteriorly extend outward from the optical axis OA. The inner peripheral surface 309 can couple to the posterior surface 305 of the anterior optical member 303 at a position that is anterior to the posterior optical member 403 and spaced radially outward relative to where the anterior optical surface 302 couples to the peripheral portion 206. In the unaccommodated state of the IOL component 200, the inner peripheral surface 309 extends anteriorly relative to the anterior surface 405 of the posterior optical member 403 and posteriorly relative to where the posterior coupler 404 connects to the optical member 403 and/or a mid-plane of the posterior optical member 403. In an accommodated state, in some embodiments, the inner peripheral surface 309 extends posteriorly relative to the posterior optical surface 402. The inner peripheral surface 309 posteriorly extends to engage with a second inclined surface 410 of the posterior member 400, described in detail below.

The inner peripheral surface 309 is offset from the posterior coupler 404 when the IOL component 200 is assembled. The inner peripheral surface 309, anterior surface or surface 407 of the posterior coupler 404, and second inclined surface 410 cooperate to define a peripheral region 211 of the closed cavity 208. The peripheral region 211 is positioned radially outward relative to the anterior optical member 303 and the posterior optical member 403.

The IOL component 200 can be configured in several ways. In one embodiment, compression of the peripheral portion 206 causes the anterior optical surface 302 to bend. At the same time, the lens body 403 can be less directly controlled by such compression. For example, the lens body 403 can be more loosely coupled to the peripheral portion 206 such that the lens body 403 is allowed to shift anteriorly with anterior bending of the anterior optical surface 302. The lens body 403 can shift posteriorly upon relaxation of the anterior optical surface 302. In some embodiments, the movement of fluid within the closed cavity 208 caused by bending of the anterior optical surface 302 shifts (e.g., pulls, pushes, or simply allows a following of) the lens body 403 anteriorly and posteriorly. In another embodiment, compression of the peripheral portion 206 directly causes the second optical surface 402 to move anteriorly. For example, the posterior coupler 404, in some embodiments, can transfer compressive forces applied to the peripheral portion 206 such that the second optical surface 402 and posterior optical member 403 are moved anteriorly. Conversely, in the absence of compressive forces being applied to the peripheral portion 206, the second optical surface 402 and the posterior optical member 403 can shift back posteriorly.

The IOL component 200 has a channel 408 that can receive joining substance 204. The channel 408 is formed in the peripheral portion 206 of the IOL component 200. As shown in FIG. 7, the channel 408 is formed in the second peripheral portion 406 of the posterior member 400 in one embodiment. Specifically, the channel 408 is formed in an anterior surface of the second peripheral portion 406 of the posterior member 400. In some variants, the channel 408 is formed in the first peripheral portion 306 of the anterior member 300. Specifically, the channel 408 can be formed in a posterior surface of the first peripheral portion 306 of the anterior member 300. The channel 408 can be an annular recess in the second peripheral portion 406 of the posterior member 400. The channel 408 can be an annular recess in the first peripheral portion 306 of the anterior member 300. The channel 408 can be an annular recess in the second peripheral portion 406 of the posterior member 400 and the first peripheral portion 306 of the anterior member 300. The position of the channel 408 can determine the location of the visible color structure (e.g., the visible joining substance 204) such that the orientation of the IOL component 200 can be detected. For example, as shown in FIG. 7, the channel 408 is disposed in the second peripheral portion 406 such that the visible joining substance 204 is closer to the posterior surface of the posterior member 400 (e.g., posterior side 401) when the IOL component 200 is viewed from the side. Although the joining substance 204 combines a connection function with providing for visibility of the orientation of the IOL component 200, these functions can be provided separately in variations. For example, the channel 408 can have walls that are modified to have a visible color and the joining substance 204 can be generally clear.

As shown in FIG. 7, the channel 408 has a trapezoidal-shaped profile. In some variants, channel 408 has a polygonal, rounded, curved, irregular, and/or other shaped profile. The channel 408 can be three-sided with an open side through which the joining substance 204 can be poured or flowed into the channel 408. The channel 408 can have two surfaces that are angled relative to each other with a third surface extending therebetween. An angle that is less than 20, 20-30, 30-40, 40-50, 50-60, 60-70, 70-80, 80-90, 90-100, 100-110, 110-120, 120-130, 130-140, 140-150, 150-160, or greater than 160 degrees can define the angle between the two surfaces. When the anterior member 300 is interfacing with the posterior member 400, the channel 408 can be enclosed by the posterior surface (e.g., cap portion 315) of the first peripheral portion 306 of the anterior member 300, which can block the flow of the joining substance 204 out of the channel 408 and/or into the closed cavity 208.

An arm 412 of the posterior member 400 extends anteriorly away from the posterior side 401 at an angle relative to the optical axis OA. The angle between the optical axis OA and the arm 412 can be less than 10, 10-20, 20-30, 30-40, 40-50, 50-60, 60-70, or greater than 70 degrees. The arm 412 defines a portion of the channel 408. Specifically, the arm 412 can define the outer surface of the channel 408. As shown in FIG. 7, the arm 412 has a decreasing thickness as the arm 412 extends away from the posterior side 400, terminating with a tip. In some variants, the arm 412 has a uniform thickness.

The exterior surface of the arm 412 can also be angled posteriorly toward the optical axis OA such that the posterior member 400 is out of contact with the base member 500 at the arm 412 when the IOL component 200 is positioned in the base member 500. Being out of contact with the base member 500 results in a reduction of load applied directly through the posterior member 400. A load applied through compression of the ciliary body is transferred to the peripheral surface 210 and thereafter to the anterior optical surface 302. This configuration enhances or focuses load transferred in this manner to bend the surface 302.

When the anterior member 300 is interfaced with the posterior member 400, the arm 412 can extend into a groove 314 (e.g., annular groove, recess), as shown in FIG. 7-1. The groove 314 is disposed in the anterior member 300. Specifically, the groove 314 is disposed in the posterior surface of the first peripheral portion 306 of the anterior member 300. The groove 314 has an angled profile in FIG. 7-1 but other profiles may be used, such as polygonal, curved (as shown in FIG. 7), irregular, and/or others. The groove 314 and the peripheral surface 210 can cooperate to define a lip 308 (e.g., circumferential lip) between them. The lip 308 extends posteriorly away from the anterior member 300. The lip 308 can be an extension of the peripheral surface 210. The arm 412 can be retained by the lip 308 such that the arm 412 does not extend beyond the peripheral surface 210.

In some variants, no groove 314 and/or circumferential lip 308 is/are present, as shown in FIG. 7-2 or FIG. 7-3. Instead, the arm 412 engages a substantially flat posterior surface of the peripheral portion 306 of the anterior member 400. Joining substance 204 can be retained by the arm 412 but excess joining substance 204 that may be squeezed out may be removed in post-processing such that the peripheral surface 210 and IOL component 200 have minimal flash or are substantially free of flash, e.g., resulting in the peripheral surface 210 being smooth and cylindrical. As shown in FIG. 7-3, the tip of the arm 412 can be removed or spaced from, e.g., positioned inward (e.g., radially inward) of the peripheral surface 210 such that a posterior surface 317 of the anterior member 300 extends between the arm 412 and the peripheral surface 210. Excess joining substance 204 squeezed out of the channel 408 can advantageously flow onto the posterior surface 317 and/or an outer surface 413 of the arm 412. The posterior surface 317 can accommodate a volume of overflow of the joining substance 204 without this overflow volume extending to the peripheral surface 210. This configuration can enable the IOL component 200 to have minimal to no flash at the peripheral surface 210. The peripheral surface 210 can be substantially free of flash even if some or all of the overflow volume is left on the posterior surface 317 and not removed. In some cases, overflow volume is removed from the posterior surface 317. Any joining substance 204 that may flow onto the peripheral surface 210 can be removed in post-processing as described above. The presence of joining substance 204 on the peripheral surface 210 can impair the accommodative response of the IOL component 200 because of increased stiffness and an uneven peripheral surface 210. Accordingly, keeping the peripheral surface 210 free or substantially free of joining substance 204 can facilitate proper accommodation of the IOL component 200. Additionally, the tip of the arm 412 can be positioned inward or can extend inwardly along the outer surface thereof relative to the peripheral surface 210 to advantageously direct compressive forces applied to the peripheral surface 210 to the anterior optical surface 302 and diminish the compressive forces transferred to the posterior optical surface 402.

Returning to FIG. 7, the IOL component 200 can have an inclined interface 212 disposed in the peripheral portion 206 between the first peripheral portion 306 and the second peripheral portion 406 when the anterior member 300 engages the posterior member 400. The inclined interface 212 can have a first inclined surface 310 (e.g., an anterior inclined surface) and second inclined surface 410 (e.g., a posterior inclined surface) that are complimentary such that they are flush when the anterior member 300 and the posterior member 400 are interfacing and/or coupled. The inclined interface 212 can be disposed between the channel 408 and the closed cavity 208 and can prevent movement of the joining substance 204 into the closed cavity 208, reducing the contamination of vision along the optical axis. The first inclined surface 310 and the second inclined surface 410 can be similarly or equally angled relative to the optical axis OA. The second inclined surface 410 can be angled relative to the innermost surface of the channel 408, which can include angles of less than 60, 60-70, 70-80, 80-90, 90-100, 100-110, 110-120, or greater than 120 degrees. The first inclined surface 310 can interface with a portion of the second inclined surface 410, which can include less than half, half, or more than half of the second inclined surface 410.

The inner peripheral surface 309, described previously herein, posteriorly extends to engage with the second inclined surface 410. The second inclined surface 410 is connected to the posterior coupler 404 such that the coupling position is posterior to and/or radially inward from where the inner peripheral surface 309 engages with the second inclined surface 410. The posterior coupler 404 and the second inclined surface 410 can be coupled at a position that is posterior to or posteriorly as the posterior optical surface 402. The second inclined surface 410 and the posterior coupler 404 can be angled relative to each other at an angle of less than 50, 50-60, 60-70, 70-80, 80-90, 90-100, 100-110, 110-120, 120-130, or greater than 130 degrees. A corner 415 can define the inner transition between the posterior coupler 404 and the second inclined surface 410. A curve 413 can define the outer transition between the posterior coupler 404 and the second inclined surface 410.

The inclined interface 212 can include a recess 311 (e.g., curved recess, groove). The first inclined surface 310 of the anterior side 300 can extend away from the recess 311. The recess 311, as shown in FIG. 7, has a curved profile, but in some variants, the recess 311 can have other shaped profiles (e.g., angled). The recess 311 can be an annular recess that circumferentially surrounds the optical axis OA. The second inclined surface 410 of the posterior side 400 can extend into a peak 411 (e.g., curve, apex, vertex). The peak 411, as shown in FIG. 7, has a curved profile, but in some variants, the peak 411 can have other shaped profiles. The peak 411 can be disposed between the second inclined surface 410 and the innermost surface of the channel 408. The peak 411 and recess 311 are complimentary such that they engage when the anterior member 300 and the posterior member 400 are interfacing, which can prevent movement of the joining substance 204 into the closed cavity 208. The peak 411 and the recess 311 can interface at a position that is anterior relative to the cap portion 315 and the channel 408. The first inclined surface 310 extends anteriorly relative to the cap portion 315 and channel 408 (e.g., anterior most portion of the channel 408) when the IOL component 200 is assembled. The first inclined surface 310 extends posteriorly relative to the channel 408 (e.g., posterior most portion of the channel 408) when the IOL component 200 is assembled. The channel 408 can be enclosed by a cap portion 315 (e.g., a posterior surface of the anterior member 300) to prevent the joining substance from entering the closed cavity 208. The cap portion 315 can be posterior relative to the recess 311 and the peak 411 when the IOL component 200 is assembled. The cap portion 315 can be positioned peripherally relative to the posterior optical member 403. The cap portion 315 can be positioned posteriorly to the anterior surface 405 of the posterior optical member 403 and anteriorly relative to the posterior optical surface 402. The cap portion 315 can be positioned radially outward relative to the recess 311, peak 411, first inclined surface 310, and second inclined surface 410.

### III. Preparation of Intraocular Lens Component

The IOL component 200 can be prepared by forming and assembling the anterior and posterior members 300, 400 thereof and then by filling the close cavity 208, as follows.

A. Formation and Coupling of Anterior and Posterior Sides

FIGS. 7A and 7B show an exploded view of the IOL component 200, showing the anterior member 300 and the posterior member 400 before coupling these components together. The anterior member 300 and the posterior member 400 can be formed separately before being joined together to form an enclosure of the IOL component 200. The anterior member 300 and the posterior member 400 can be formed by flowing a material into shells (e.g, molds). Specifically, the anterior member 300 and posterior member 400 are each formed by flowing material between two opposable shell portions. Once the anterior member 300 is formed, the shell portion that is coupled to the posterior surface of the anterior member 300 can be removed, leaving the anterior member 300 coupled to the shell portion engaging the anterior surface of the anterior member 300. Similarly, once the posterior member 400 is formed, a shell (e.g., mold) portion that is coupled to the anterior surface of the posterior member 400 is removed, leaving the posterior member 400 coupled to the shell portion engaging the posterior surface of the posterior member 400. Any excess material coupled to the anterior member 300 or posterior member 400 can be removed while the anterior member 300 and posterior member 400 are coupled to their respective shell portions. Excess material coupled to the anterior member 300 or posterior member 400 from formation of the member 300, 400 can be removed after coupling the anterior member 300 and posterior member 400 together to form the IOL component 200. Such a process can also remove excess joining substance 204, as discussed further below.

After formation of the anterior member 300 and the posterior member 400, a joining substance 204 is used to join (e.g, couple, adhere, or secure along a seam) the anterior member 300 to the posterior member 400. A joining substance 204 can be applied to one or both of an anterior surface of the second peripheral portion 406 or a posterior surface of the first peripheral portion 306 for coupling. In some variants, a channel 408 (e.g., joining channel, annular recess) disposed and/or formed in one or both of the first peripheral portion 306 and the second peripheral 406 is filled with or partially filled with the joining substance 204. The channel 408 can be a continuous structure that can aid in confirming orientation of the IOL component 200 and proper integration of the IOL component 200 with the base member 500. As shown in FIG. 7C, the joining substance 204 can be flowed into the channel 408 formed in the anterior surface of the second peripheral portion 406 of the posterior side 400. A dye and/or pigment can be added to the joining substance 204 such that a visible color (e.g., yellow) mixed with the joining substance 204 can be detected, which can advantageously enable the verification of the orientation of the IOL component 200 as described herein. In some techniques, one or more walls of the channel 408 can be colored which allows the joining substance 204 to be clear. Also, one or more walls of the channel 408 can be made to appear a first color and the joining substance 204 can be made to appear a second color.

As shown in FIG. 7D, the first member 300 (e.g., posterior member) and the second member 400 (e.g., posterior member) are joined (e.g., bonded, adhered, coupled) together to form an enclosure of the IOL component 200 after the application of the joining substance 204. In a preferred variant, the shell portion engaging the anterior surface of the anterior member 300 and the shell portion engaging the posterior surface of the posterior member 400 engage (e.g., mate, couple, interface) with each other while the first member 300 and the second member are bonded together, facilitating the correct alignment between the first member 300 and the second member 400 and improved bonding. In some variants, a periphery portion of the shell portions engage during bonding while the anterior member 300 and the posterior member 400 occupy the central portion of the shell portions.

The channel 408 is enclosed to block the flow of the joining substance 204 into the closed cavity 208. More specifically, enclosing the channel 408 can include engaging the inclined interface 212 formed between the second inclined surface 410 of the second peripheral portion 406 and the first inclined surface 310 of the first peripheral portion 306. A cap portion 315 of the posterior surface of the first peripheral portion 306 covers the open side of the channel 408 to further enclose the channel 308. In some variants, the peak 411 and recess 311 engage between the channel 408 and the closed cavity 208 to further prevent joining substance 204 from flowing into the closed cavity 208. The peak 411 and recess 311 can engage at an anterior position relative to the cap portion 315 of the posterior surface of the first peripheral portion 306 that covers the channel 306.

The combination of the recess 311 and peak 411 along with the inclined interface 212 provide multiple barriers to migration or flow of the joining substance 204 toward the closed cavity 208. The enclosure of the IOL component 200 can be formed by coupling the anterior member 300 to the posterior member 400 with the posterior member 400 below the anterior member 300. The joining substance 204 will thus have to flow up toward the peak 411 to flow over toward to closed cavity 208. Such flow will be impeded by the recess 311 which caps the peak 411. Further, the interface 212 will involve direct contact between the inclined surfaces 310, 410. Such direct contact will reduce, minimize, or eliminate flow paths from the peak 411 capped by the recess 311 toward the closed cavity 208.

The closed cavity 208 is formed between the anterior optical surface 302 and the posterior optical surface 402 by forming a seal between the first peripheral portion 306 of the anterior member 300 and the second peripheral portion 406 of the posterior member 400. The closed cavity 208 can be bounded by a posterior surface 305 of the anterior member 300 and an anterior surface 405 of the posterior member 400. The posterior surface 305 and an inclined inner periphery 309 of the first peripheral portion 306 form a concave recess 307 in which a portion of the posterior member 400, e.g., the anterior surface 405 of the posterior member 400 and in some cases a majority of a bi-convex optic of the posterior member 400 can be disposed in an at-rest condition. The closed cavity 208 can be bounded by an anterior surface or surfaces 407 of the posterior coupler 404 and the anterior surface 405 of the posterior member 400. The anterior surface or surfaces 407 of the posterior coupler 404 and the anterior surface 405 of the posterior member 400 can form a convex projection at least partially and in some cases mostly received in the concave recess 307 of the anterior member 300. The joining substance 204 couples the first peripheral portion 306 and the second peripheral portion 406 together such that the joining substance 204, which can include cooperation with the inclined interface 212, form a seal that prevents fluid and/or gel to be disposed in the closed cavity 208 (as discussed herein) from escaping the IOL component 200. The visible quality of the joining substance 204 enables visual confirmation that the closed cavity is substantially free or entirely free of the joining substance 204.

Excess joining substance 204 and/or material from the formation of the anterior member 300 and the second member 400 can result in flash forming on the peripheral portion 206 of the formed IOL component 200. Flash can be removed (e.g., cut, ground, smoothed) from the peripheral portion 206 such that the peripheral surface 210 is smooth. Providing a smooth peripheral surface 210, e.g., free of flash, can enhance or maintain responsiveness of the IOL component 200 to ocular forces which can be relatively small. Providing a smooth peripheral surface 210, e.g., free of flash, can reduce, minimize, or eliminate resistance to deformation of the anterior optical surface 302 by ocular forces.

B. Method of Filling and Closing the Closed Cavity

FIG. 8 shows the enclosed chamber of the IOL component 200 assembled with the anterior member 300 coupled to the posterior member 400. The closed cavity 208 is disposed between the anterior optical surface 302, posterior optical surface 402, and the peripheral portion 206. Stated differently, the peripheral portion 206 formed by coupling the first peripheral portion 306 of the anterior member 300 to the second peripheral portion 406 of the posterior member 400 form an empty closed cavity 208 therebetween that can hold a liquid material (e.g., optical fluid, fluid, lens oil) or gel.

The enclosed chamber 208 can be accessible from the peripheral surface 210 of the peripheral portion 206, for example by a port 220 extending toward the closed cavity 208. In other embodiments, another optically peripheral structure of the IOL component 200 can include the port 220. The port 220 has a first end 222 (e.g., opening, outward end) disposed at the peripheral surface 210 and a second end 224 (e.g., wall, solid wall) disposed within the peripheral portion 206 between the first end 222 and the closed cavity 208. A solid and continuous expanse of material can extend between the second end 224 of the port 220 and the closed cavity 208. The port 220 can have a periphery with any shape, e.g., a circular shape. In some variants, other shaped peripheries can be employed. The port 220 can have a consistent size (e.g., diameter) from the first end 222 to the second end 224, or in some variants, the port 220 can have a varying size (e.g., a diameter that decreases in the first end 222 to second end 224 direction). In some cases, an access pathway is provided between the second end 224 and the closed cavity 208, e.g., a self-sealing structure or a structure that can seal to an injector.

In one technique, a tubular member 700 can be advanced into and through the port 220 such that the tubular member 700 extends into the closed cavity 208. Advancing the tubular member 700 into and through the port 220 can include piercing the second end 224 to provide access to the closed cavity 208. FIG. 8 shows that in one at-rest state the bi-convex optic 403 of the IOL component 200 may be aligned with the port 220. This can result from the compact arrangement resulting from the fact that the bi-convex optic is received in the concave recess 307. While it is desirable to provide a compact arrangement along the optical axis of the eye when the IOL component 200 is implanted, it is preferred to not contact a sharp end of the tubular member 700 with the anterior surface 405 of the bi-convex optic 403. Accordingly, a compressive force is applied to the peripheral surface 210 causing further separation and/or movement of the first optical surface 302 and/or second optical surface 402 (e.g., the anterior surface 405 of the posterior member 400) such that the posterior surface 305 of the anterior member 300 and/or anterior surface 405 of the posterior member 400 are not in the insertion path 704 of the tubular member 700, as shown in FIG. 8A. This advantageously enables the tubular member 700 to be inserted into the closed cavity 208 without puncturing or scratching the first optical surface 302, the posterior surface 305 of the anterior member 300, the anterior surface 405 of the posterior member 400 and/or second optical surface 402, which could compromise the IOL component 200. In some variants, the closed cavity 208, first optical surface 302, the posterior surface 305 of the anterior member 300, the anterior surface 405 of the posterior member 400, and/or second optical surface 402 are sized and shaped such that these surfaces are not in the insertion path 704 of the tubular member 700 absent a compressive force.

Liquid material 230 (e.g., optical fluid, fluid, lens oil) or a gel is dispensed in the closed cavity 208 via the tubular member 700 to provide a continuous expanse of the liquid material 230 between the anterior optical surface 302 and the posterior optical surface 402, or between the posterior surface 305 of the anterior member 300 and the anterior surface 405 of the posterior member 400. As shown in FIG. 8A, a tip 702 of the tubular member 700 is positioned at a side of the closed cavity 208 that is opposite the port 200. The tip 702 is withdrawn (e.g., moved towards the port 220) while liquid material 230 is flowed into the closed cavity 208, as shown in FIGS. 8B and 8C. As shown in FIG. 8D, the tubular member 700 is withdrawn from the closed cavity 208 through the port 220. The tubular member 700, in some variants, is not withdrawn from the closed cavity 208 until the entire closed cavity 208 is filled with the liquid material 230. A plug 202 is formed and/or placed in the port 220 after withdrawing the tubular member 700 from the port 220. In some variants, a conduit 226, illustrated in FIG. 9, created by the tubular member 700 through the peripheral portion 206 seals shut after removal of the tubular member 700, and in some variants, the plug 202 seals the conduit 226 such that liquid material 230 does not escape the closed cavity 208. In some cases, the conduit 226 seals shut and the plug 202 provides an extra measure of retentive security for the material that flows into the closed cavity 208.

In some variants, a channel is formed between the second end 224 of the port 220 and the closed cavity 208 prior to insertion of the tubular member 300. The pre-formed channel provides access to the closed cavity 208 by a tubular member 700 (e.g., a syringe) with less resistance and greater precision and control. The tubular member 700 can be inserted through the channel and into the closed cavity 208 such that liquid material (e.g., fluid, optical fluid, gel) flows into the closed cavity 208 via the tubular member 700. The channel can be self-sealing in the absence of the tubular member 700 such that the channel closes preventing liquid material from escaping the closed cavity 208 in the absence of the tubular member 700. The channel can be blocked by the plug 202, as discussed above.

In some variants, a marker 240 is positioned on the tubular member 700, as shown in FIG. 8D, indicating when the tip 702 of the tubular member 700 is properly positioned at the side of the closed cavity 208 opposite the port 220 such that advancement of the tubular member 700 should cease. This can advantageously signal that advancement should stop before the tip 702 pierces the peripheral portion 206 opposite the port 220. In some variants, the marker 240 is a plug that can be positioned in the port 220 as the tip 702 of the tubular member 700 is withdrawn from the port 220. The combined plug/marker can be pre-loaded onto the tubular member 700 of a syringe. An aperture through the pre-mounted marker-plug can seal shut once the tubular member 700 is removed, effectively fully enclosing the port 220.

### IV. Assembly of Intraocular Device

FIG. 10A illustrates the IOL component 200 assembled with the base member 500. As explained elsewhere herein, the IOL component 200 can be assembled to the base member 500 inside or outside the eye of a patient. As assembled, a peripheral portion of the IOL component 200 can be positioned behind the retention features 502 to retain the IOL component 200 within the base member 500. To further ensure retention, the assembly which can be formed as an accommodating IOL can have one or more surfaces configured to enhance adherence. For example, the IOL component 200 can have one or more surfaces that adhere to the surface(s) of the base member 500. For example, the anterior surface of the first peripheral portion 306, a posterior surface of the second peripheral portion 406, and/or a peripheral surface 210 of the IOL component 200 can have adhesive qualities (e.g., tacky, sticky) that adhere to the surfaces of the base member 500 upon contact therewith. In some embodiments, the base member 500 can have one or more surfaces that adhere to the surface(s) of the IOL component 200. For example, the posterior surface 503 of the retaining feature 502, the radially inward facing surface 507, and/or the table 505 of the base member 500 can have adhesive qualities (e.g., tacky, sticky) that adhere to the surfaces of the IOL component 200 upon contact therewith. In some cases, these or other surfaces of the base member 500 and the IOL component 200 can have adhesive qualities as described herein.

As shown, the first peripheral portion 306 interfaces with the posterior surface 503 of the retaining feature 502, an radially inward facing surface 507 interfaces with the peripheral surface 210, and/or a table 505 interfaces with the posterior surface of the second peripheral portion 406 when the IOL component 200 is assembled with the base member 500. All or some of the forenamed surfaces can have adhesive qualities that can improve retention of the IOL component 200 within the base member 500.

In one embodiment the anterior surface of the first peripheral portion 306, a posterior surface of the second peripheral portion 406, and/or a peripheral surface 210 of the IOL component 200 can have adhesive characteristic around the entirety thereof. In one variation the adhesive quality of or on one or more of these surfaces can be spaced apart about the periphery of the component 200. Where the adhesive quality is spaced apart an expanse of the surface between the adhesive areas can be less or non-adhesive. By providing non- or less adhesive surface between the high or higher adhesive surfaces the IOL component 200 can be configured to be rotated within the base member 500 to a pre-defined position. As such the IOL component 200 can be placed in the base member 500 and then rotated until the higher adhesive surfaces of the IOL component 200 reaches the location of the retaining features 502. This facilitates achieving a pre-defined rotational position between the IOL component 200 and the base member 500. This can be useful for providing for correction of astigmatism or other rotationally varied optical power.

One advantage of making the interfacing surfaces of the base member 500 more adherent and allowing the surface of the IOL component to be less adherent relates to one method of implanting these components. In particular it is desired to minimize the invasiveness of the procedure. Injectors can be used to implant each of these components. The surfaces of the retaining features 502, the radially inward facing surface 507 and the table 505 mentioned above can be folded into a central position away from lumens of an injector. The surfaces of the IOL component 200 described as optionally being made more adherent may be exposed to walls of the lumen of the injector. As such, positioning the more adherent surface on the IOL component 200 may result in a higher injection force being required. So maintaining exterior surfaces of the IOL component 200 as low friction can be advantageous in some embodiments.

The surface area of the interfacing surfaces of the IOL component 200 and/or the base member 500 can be increased to facilitate improved adhesion between the surfaces of the IOL component 200 and the surfaces of the base member 500. Specifically, the surface area of the anterior surface of the first peripheral portion 306, a posterior surface of the second peripheral portion 406, and/or a peripheral surface 210 of the IOL component 200 can be increased to facilitate improved adhesion. Similarly, the surface area of the posterior surface 503 of the retaining feature 502, the radially inward facing surface 507, and/or the table 505 of the base member 500 can be increased to facilitate improved adhesion.

The surface area of the surfaces of the IOL component 200 and/or base member 500 can be increased with roughening. Roughening can be accomplished during the molding of the IOL component 200 and/or base member 500. Roughening can also be accomplished after the molding of the IOL component 200 and/or base member 500. In some embodiments, surfaces of the IOL component 200 and/or base member 500 are treated after molding to achieve a desired level of roughness (e.g., surface area). In some embodiments, the mold surfaces that form the surfaces of the IOL component 200 and/or base member 500 are roughened to form surfaces with a desired level of roughness (e.g., surface area). The mold surfaces can be roughened by electrical discharge machining (EDM), machining (milling and lathing), sand blasting, stamping, and/or other suitable techniques.

As illustrated in FIG. 10B, improved retention can also be accomplished by inducing and/or enhancing a normal force (indicated by the arrow N) on the IOL component 200 with the one or more retention features 502 (e.g., lens retention members, tabs, protrusions, retainers, etc.) in full assembly. For example, the one or more retention features 502 can be resilient with a radially inner portion or end biased posteriorly, prior to full assembly, into a receiving space 511 that receives the IOL component 200 in full assembly. The receiving space 511 can be between the retaining feature 502 (e.g., the posterior surface 503 of the retaining feature 502) and the table 505 (e.g., anterior surface of the table 505). In some embodiments, the one or more retention features 502 can be angled, curved, and/or otherwise disposed posteriorly when not subject to an anterior force, such as a lifting force L.

During assembly, the one or more retention features 502 can be lifted (e.g., deflected, rotated) anteriorly, as indicated by the lifting force arrow L, to a new position 502′ that facilitates placing the IOL component 200 within the receiving space 511 and behind the one or more retention features 502. Upon removing the lifting force L, the one or more retention features 502 can apply the normal force N to the IOL component 200. Specifically, the one or more retention features can apply the normal force N to the anterior surface of the first peripheral portion 306. The normal force N can also be applied to the posterior surface of the second peripheral portion 406. This normal force N increases friction between the IOL component 200 and the base member 500 to facilitate improved retention. In some embodiments, the normal force N results in the peripheral portion 206 of the IOL component 200 being compressed (e.g., clamped, pinched) between the one or more retention members 502 and the one or more tables 505 without negatively impacting the accommodative responses of the IOL component 202.

Providing a posteriorly biased and resilient retention feature 502 can facilitate confirming that an IOL including the IOL component 200 and the base member 500 have been assembled in a pre-defined rotational position. For example, where an IOL including the IOL component 200 and the base member 500 is configured to mitigate astigmatism the IOL component 200 should be positioned at a pre-defined rotational position. The IOL component 200 can be configured to stop rotating when a portion thereof reaches the location of a resilient retention feature 502. For example, a radially oriented notch 300N can be provided on an anterior surface of the anterior member 300 of the IOL component 200, as shown in FIGS. 10C and 10D. The notch 300N can be provided on an anterior surface of the peripheral portion 206 (e.g., the first peripheral portion 306′). The notch 300N can be wider than the circumferential width of the resilient retention feature 502. As such, the IOL component 200 rotates into alignment with the resilient retention feature 502 such that the feature 502 can fall into the notch 300N. Walls of the notch 300N can bound the resilient retention feature 502 preventing more than minimal rotation within the notch 300N.

As illustrated in FIG. 10E, the retention feature (e.g., tab, protrusion) 502″ can flex (e.g., deflect) to enable the IOL component 200 to be inserted into the receiving space 511 and remain flexed (e.g., deflected) anteriorly with the IOL component 200 positioned within the receiving space 511. A thickness of the IOL component 200 can be greater than a thickness of the receiving space 511, such that an anterior surface of the IOL component 200 pushes against the retention feature 502″ causing the retention feature 502″ to remain in an anteriorly flexed (e.g., deflected) position when the IOL component 200 is positioned within the receiving space 511. More specifically, an anterior-posterior thickness 207 of the peripheral portion 206 of the IOL component 200 can be greater than an anterior-posterior thickness 513 of the receiving space 511 such that the retention feature 502″ deflects anteriorly with the IOL component 200 positioned within the receiving space 511. The anterior-posterior thickness 207 can be the distance between the anterior and posterior surfaces of the peripheral portion 206. The anterior-posterior thickness 511 can be the distance between the posterior surface 503 of the retaining feature 502″ and the table 505. In some embodiments, the relatively larger anterior-posterior thickness 207 of the IOL component 200 can inhibit contact between the peripheral surface 210 and the radially inward facing surface 507. In some embodiments, the relatively larger anterior-posterior thickness 207 of the IOL component 200 does not inhibit contact between the peripheral surface 210 and the radially inward facing surface 507. As explained elsewhere herein, the retention feature 502″ can be deflected anteriorly during assembly, to facilitate placing the IOL component 200 within the receiving space 511 and behind the one or more retention features 502″. Once assembled, the IOL component 200 can push against the one or more retention features 502″ such that the one or more retention features 502″ remains in an anteriorly deflected position.

### Terminology

As used herein, the relative terms “proximal” and “distal” shall be defined from the perspective of the medical professional. Thus, proximal refers to the direction of the physician and distal refers to the direction of the eye when the surgeon is operating.

For expository purposes, the term “transverse” as used herein is defined as a direction generally perpendicular to the longitudinal axis of the assembly, unless otherwise specified.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments.

The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

The terms “approximately,” “about,” “generally,” and “substantially” as used herein represent an amount close to the stated amount that still performs a desired function or achieves a desired result. For example, the terms “approximately,” “about,” “generally,” and “substantially” may refer to an amount that is within less than 10% of the stated amount, as the context may dictate.

The ranges disclosed herein also encompass any and all overlap, sub-ranges, and combinations thereof. Language such as “up to,” “at least,” “greater than,” “less than,” “between” and the like includes the number recited. Numbers preceded by a term such as “about” or “approximately” include the recited numbers. For example, “about four” includes “four”

Any methods disclosed herein need not be performed in the order recited. The methods disclosed herein include certain actions taken by a practitioner; however, they can also include any third-party instruction of those actions, either expressly or by implication. For example, actions such as “distally moving a locking element” include “instructing distal movement of the locking element.”

Although certain embodiments and examples have been described herein, it will be understood by those skilled in the art that many aspects of the humeral assemblies shown and described in the present disclosure may be differently combined and/or modified to form still further embodiments or acceptable examples. All such modifications and variations are intended to be included herein within the scope of this disclosure. A wide variety of designs and approaches are possible. No feature, structure, or step disclosed herein is essential or indispensable.

Some embodiments have been described in connection with the accompanying drawings. However, it should be understood that the figures are not drawn to scale. Distances, angles, etc. are merely illustrative and do not necessarily bear an exact relationship to actual dimensions and layout of the devices illustrated. Components can be added, removed, and/or rearranged. Further, the disclosure herein of any particular feature, aspect, method, property, characteristic, quality, attribute, element, or the like in connection with various embodiments can be used in all other embodiments set forth herein. Additionally, it will be recognized that any methods described herein may be practiced using any device suitable for performing the recited steps.

For purposes of this disclosure, certain aspects, advantages, and novel features are described herein. It is to be understood that not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Moreover, while illustrative embodiments have been described herein, the scope of any and all embodiments having equivalent elements, modifications, omissions, combinations (e.g., of aspects across various embodiments), adaptations and/or alterations as would be appreciated by those in the art based on the present disclosure. The limitations in the claims are to be interpreted broadly based on the language employed in the claims and not limited to the examples described in the present specification or during the prosecution of the application, which examples are to be construed as non-exclusive. Further, the actions of the disclosed processes and methods may be modified in any manner, including by reordering actions and/or inserting additional actions and/or deleting actions. It is intended, therefore, that the specification and examples be considered as illustrative only, with a true scope and spirit being indicated by the claims and their full scope of equivalents.

What is claimed is:

1. An intraocular lens component, comprising:
an anterior side comprising an anterior optical surface disposed across an optical axis of the lens component;
a posterior side comprising a posterior optical surface disposed across the optical axis;
a peripheral portion having an anterior portion coupled to the anterior side, a posterior portion coupled to the posterior side, and a joining channel disposed in one or both of the anterior portion and the posterior portion; and
a joining substance disposed in the joining channel to bond the posterior portion of the peripheral portion to the anterior portion of the peripheral portion.

2. The intraocular lens component of claim 1, wherein the joining substance includes a pigment or dye configured to facilitate visualization of an orientation of an intraocular lens.

3. The intraocular lens component of claim 2, wherein the pigment or dye is closer to the anterior side or posterior side.

4. The intraocular lens component of any of the preceding claims, wherein the joining substance forms a continuous feature in the peripheral portion that is configured to visually verify assembly of an intraocular lens when viewed from the anterior side.

5. The intraocular lens component of claim 4, wherein the continuous feature is an annular structure that surrounds the optical axis of the lens component.

6. The intraocular lens component of claim 4, wherein the continuous feature is configured to be visually disrupted under a retaining feature of the intraocular lens to visually verify the assembly of the intraocular lens when viewed from the anterior side.

7. The intraocular lens component of claim 1, further comprising an inclined interface disposed in the peripheral portion between the posterior portion and the anterior portion.

8. The intraocular lens component of claim 7, wherein the inclined interface comprises an anterior inclined surface and a posterior inclined surface, the inclined interface being disposed between the joining channel and a closed cavity.

9. The intraocular lens component of claim 8, wherein the closed cavity is between the anterior optical surface and the posterior optical surface.

10. The intraocular lens component of any of the preceding claims, wherein the intraocular lens component comprises a material that is configured to adhere to a surface of a base member upon contact.

11. The intraocular lens component of any of the preceding claims, wherein an anterior surface of the anterior portion, a posterior surface of the posterior portion, and a peripheral surface of the peripheral portion are rough to facilitate improved adhesion to the surface of the base member upon contact.

12. The intraocular lens component of claim 11, wherein the peripheral surface of the peripheral portion is rough to facilitate improved adhesion to the surface of the base member upon contact.

13. An intraocular lens (IOL) device comprising the intraocular lens component of any of the preceding claims and a base member comprising a haptic configured to engage a capsular bag of an eye of a patient and one or more retention features, wherein the base member comprises a material that is configured to adhere to a surface of the IOL component upon contact.

14. The IOL device of claim 13, wherein the base member comprises a radially inward facing wall, posterior surface of the one or more retention members, and/or one or more tables, wherein a receiving space is adjacent the radially inward facing wall and/or between the one or more retention members and the one or more tables, wherein the receiving space is configured to receive the IOL component, and wherein the radially inward facing wall, posterior surface of the one or more retention members, and/or one or more table are rough to facilitate improved adhesion to surface(s) of the IOL component upon contact.

15. A method of assembling a fluid lens, comprising:
providing an anterior member comprising an anterior optical surface and a first peripheral portion;
providing a posterior member comprising a posterior optical surface and a second peripheral portion;
applying a joining substance to one or both of an anterior surface of the second peripheral portion or a posterior surface of the first peripheral portion;
coupling the anterior surface of the second peripheral portion with the posterior surface of the first peripheral portion using the joining substance; and
forming a closed cavity between the anterior optical surface and the posterior optical surface by forming a seal between the first peripheral portion and the second of the peripheral portion.

16. The method of claim 15, wherein applying the joining substance comprises flowing a liquid material into a channel formed in the anterior surface of the second peripheral portion or into a channel formed in the posterior surface of the first peripheral portion.

17. The method of claim 15, wherein applying the joining substance comprises flowing a liquid material into a channel formed in the anterior surface of the second peripheral portion.

18. The method of claim 16, further comprising enclosing the channel to block flow of the joining substance into the closed cavity.

19. The method of claim 18, wherein enclosing the channel includes engaging an inclined interface formed between a second inclined surface of the second peripheral portion and a first inclined surface of the first peripheral portion.

20. The method of any of claims 15-19, further comprising confirming that the closed cavity is substantially free of the joining substance.

21. The method of any of claims 15-19, further comprising detecting a visible color mixed with the joining substance.

22. A method of preparing a fluid lens component, comprising:
providing a fluid lens component comprising a closed cavity disposed between an anterior optical member, a posterior optical member, and a peripheral portion, the peripheral portion comprising a peripheral surface having a port extending toward the closed cavity, the anterior optical member having a first surface on an anterior side of the fluid lens and a second surface opposite the first surface, the second surface at least partially bounding the closed cavity;
advancing a tubular member into and through the port such that the tubular member extends into the closed cavity;
dispensing an optical fluid in the closed cavity to provide a continuous expanse of the optical fluid adjacent to the second surface of the anterior optical member; and
withdrawing the tubular member from the port.

23. The method of claim 22, wherein the fluid lens component comprises a solid wall disposed between an inward end of the port and the closed cavity, and wherein advancing the tubular member comprises piercing the solid wall to provide access to the closed cavity through the port.

24. The method of claim 22 or 23, wherein dispensing the optical fluid comprises positioning a tip of the tubular member at a side of the closed cavity opposite the port and withdrawing the tip as the fluid is flowing into the closed cavity.

25. The method of any of claims 22-24, further comprising forming a plug in the port after withdrawing the tubular member from the port.

26. An intraocular lens component, comprising:
an anterior side comprising an anterior optical surface disposed across an optical axis of the lens component;
a posterior side comprising a posterior optical surface disposed across the optical axis;
a peripheral portion coupled to the anterior side and the posterior side, the peripheral portion, anterior side, and posterior side forming a closed cavity therebetween; and
a port having a first end disposed at a peripheral surface of the peripheral portion and a second end disposed within the peripheral portion between the first end and the closed cavity.

27. The intraocular lens component of claim 26, wherein a solid and continuous expanse of material extends between the second end of the port and the closed cavity.

28. The intraocular lens component of claim 26 or 27, further comprising a channel formed between the second end of the port and the closed cavity for access to the closed cavity by a syringe, the channel being self-sealing in the absence of the syringe.

29. The intraocular lens component of any of claims 26-28, further comprising an optical fluid disposed in the closed cavity.

30. The intraocular lens component of any of claims 26-29, further comprising a plug member disposed in the port.

31. An intraocular lens device comprising:
an intraocular lens (IOL) component comprising an anterior side having an anterior optical surface disposed across an optical axis of the intraocular lens component, a posterior side comprising a posterior optical surface disposed across the optical axis, and a peripheral portion coupled to the anterior side and the posterior side, wherein a fluid-filled cavity extends between the anterior optical surface, posterior optical surface, and peripheral portion;
a base member comprising a haptic configured to engage a capsular bag of an eye of a patient and one or more resilient retention features having an inner end biased posteriorly into a receiving space configured to receive the (IOL) component, wherein the one or more resilient retention features is configured to be moved in an anterior direction to facilitate placing the IOL component into the receiving space of the base member and to be released to be moved toward an anterior surface of the peripheral portion of the IOL component.

32. The intraocular lens of claim 31, wherein the resilient retention feature is configured, upon being released after the IOL component is disposed in the base member, to apply a normal force on the anterior surface of the peripheral portion of the IOL component.

33. The intraocular lens of claim 31 or 32, wherein the IOL component comprises a notch configured to receive one or more of the resilient retention features when the IOL component is at a pre-defined rotational position relative to the base member.

34. The intraocular lens of any of claims 31-33, wherein the IOL component comprises an anterior-posterior thickness that is larger than an anterior-posterior thickness of the receiving space such that the IOL component causes the one or more resilient retention features to flex anteriorly upon assembly of the IOL component within the base member.

35. A method of assembly an intraocular lens (IOL) device within an eye of a patient, comprising:
inserting a base member comprising a base optic and one or more resilient retention features that have an inner end biased posteriorly toward the base optic into an eye of a patient;
moving the one or more resilient retention features anteriorly relative to a free state of the resilient retention feature;
inserting an intraocular lens (IOL) component into a receiving space between the one or more resilient retention features and an opposite portion of the base member; and
releasing the one or more resilient retention features such that the one or more resilient retention features contacts an anterior surface of the IOL component.

36. The method of claim 35, wherein following releasing the one or more resilient retention features results in the one or more resilient retention feature(s) moving into a notch in the IOL component.

37. The method of claim 35 or 36, wherein the one or more resilient retention features applies a normal force to the anterior surface of the IOL component.

38. The method of any of claims 35-37, further comprising contacting a peripheral surface of the IOL component with a radially inward facing surface of the base member.

39. The method of any of claims 35-38, wherein the anterior surface of the IOL component anteriorly deflects the one or more resilient retention features.
